(19) United States
(12) Patent Application Publication
(10) Pub. No.: US 2008/0234472 A1
(43) Pub. Date: Sep. 25, 2008

(54) Sucralose production method

(76) Inventors: Duane Leinhos, McIntosh, AL (US); John Kerr, South Croyden (GB); Bob Jansen, Portela LRS (PT); Sebastien Camborieux, Surrey (GB); Peter Lloyd-Jones, Lancashire (GB); Anthony Baiada, Essex (GB)

Correspondence Address: RATNERPRESTIA, P.O. BOX 1596, WILMINGTON, DE, 19899, US

(21) Appl. No.: 12/009,455

(22) Filed: Jan. 18, 2008

Related U.S. Application Data
(60) Provisional application No. 60/881,292, filed on Jan. 19, 2007.

(51) Int. Cl. C07H 1/00

(52) U.S. Cl. 536/18.5

(57) ABSTRACT

There is provided a method for the production of sucralose from a feed stream resulting from the chlorination of a sucrose-6-acylate in a reaction vehicle, said feed stream comprising a sucralose-6-acylate, the reaction vehicle, and by-products including high molecular weight coloured material, said method comprising:
deacylation of the sucralose-6-acylate by treatment with a base to afford sucralose, and, before or after said deacylation,
removal of the reaction vehicle and
isolation of the sucralose
   characterised in that
immediately before the removal of the reaction vehicle, the reaction stream is subjected to a precipitation step comprising treatment with a metal or ammonium hydroxide and carbon dioxide to form a precipitate of the corresponding metal or ammonium carbonate in which at least a portion of said high molecular weight coloured material is trapped, followed by separation of said precipitate.

### CROSS REFERENCE TO RELATED APPLICATIONS

This application claims priority benefit of U.S. Provisional Patent Application No. 60/881,292, filed Jan. 19, 2007, the entire disclosure of which is expressly incorporated by reference herein.

The present invention relates to an improved method for the production of sucralose. In particular, the present invention relates to an improved method for producing sucralose from a crude reaction mixture comprising a sucralose-6-acylate obtained from the chlorination of a sucrose-6-acylate in a reaction vehicle without isolation of the sucralose-6-acylate.

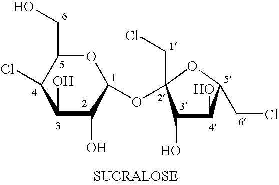

### BACKGROUND OF THE INVENTION

EP 0708110 discloses a method for the production of sucralose from the reaction mixture resulting from the chlorination of a sucrose-6-acylate in a tertiary amide reaction vehicle, without isolation of the sucralose-6-acylate intermediate, comprising deacetylation of the sucralose-6-acylate before or after removal of the tertiary amide reaction vehicle, and then isolation of the sucralose. The removal of the tertiary amide (which is usually DMF) is carried out by steam stripping.

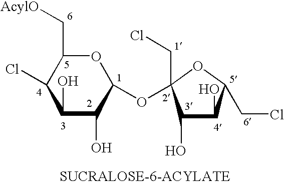

EP 0708110 states that it is preferred to perform the deacetylation after the removal of the tertiary amide reaction vehicle, because otherwise, during the deacetylation step, base-catalysed decomposition of the tertiary amide occurs. This hinders the subsequent isolation of the sucralose, and also means that the tertiary amide cannot be efficiently recovered and recycled.

### SUMMARY OF THE INVENTION

In one aspect, the invention provides a method for the production of sucralose from a feed stream resulting from the chlorination of a sucrose-6-acylate in a reaction vehicle. The feed stream includes a sucralose-6-acylate, the reaction vehicle, and by-products including high molecular weight coloured material. The method includes:
deacylating the sucralose-6-acylate by treatment with a base to afford sucralose, and, before or after the deacylation,
removing the reaction vehicle and
isolating the sucralose.

Immediately before the removal of the reaction vehicle, the reaction stream is subjected to a precipitation step including treatment with a metal or ammonium hydroxide and carbon dioxide to form a precipitate of the corresponding metal or ammonium carbonate in which at least a portion of the high molecular weight coloured material is trapped, followed by separation of the precipitate.

### DETAILED DESCRIPTION OF THE INVENTION

The present inventors have discovered that, following the chlorination reaction, when the reaction vehicle is removed, insoluble high molecular weight coloured material is produced. This represents a significant problem when the reaction vehicle is removed, because then the high molecular weight coloured material deposits in the apparatus used for removing the reaction vehicle. This is disadvantageous because some of the desired product is trapped in the deposit, resulting in reduced yields, and also because production must be halted from time to time to clean the apparatus. The reaction vehicle can be removed in a number of ways, for example by steam stripping, or by using an agitated thin film dryer or a spray drier. Steam stripping is preferred.

Without wishing to be bound by theory, it is believed that the insoluble high molecular weight coloured material results from polymeric by-products which are formed during the chlorination of the sucrose-6-acylate. These by-products lead to the deposits of the insoluble high molecular weight coloured material during the removal of the reaction vehicle, partly because they are less soluble in water than in DMF, and therefore precipitate as the DMF is removed, and partly because the polymeric chains grow during the removal of the reaction vehicle, giving higher molecular weight polymers with reduced solubility.

Furthermore, it appears that the production of high molecular weight coloured by-products is associated with the chlorination of sucrose-6-acylates generally, irrespective of the chlorination agent and/or solvent employed, and therefore their removal is a problem in all synthetic routes to sucralose that proceed via the chlorination of a sucrose-6-acylate.

Therefore, it is an aim of the present invention to provide a method for the removal of high molecular weight coloured material formed as a by-product in the chlorination of sucrose-6-acylates.

A more specific aim of the present invention is to reduce the deposition of the insoluble high molecular weight coloured material when the reaction vehicle is removed following the chlorination of sucrose-6-acylates.

According to the present invention, it has been found that treating the reaction mixture with a metal or ammonium hydroxide and carbon dioxide results in precipitation of the corresponding metal or ammonium carbonate, and that the high molecular weight coloured by-products are trapped in the precipitate, which can then be filtered off. This reduces the deposition of the insoluble coloured high molecular weight material when the reaction vehicle is removed, and can increase the sucralose yield.

According to the present invention, there is provided a method for the production of sucralose from a feed stream resulting from the chlorination of a sucrose-6-acylate in a reaction vehicle, said feed stream comprising a sucralose-6-acylate, the reaction vehicle, and by-products including high molecular weight coloured material, said method comprising:
deacylation of the sucralose-6-acylate by treatment with a base to afford sucralose, and, before or after said deacylation,
removal of the reaction vehicle and
isolation of the sucralose
   characterised in that
immediately before the removal of the reaction vehicle, the reaction stream is subjected to a precipitation step comprising treatment with a metal or ammonium hydroxide and carbon dioxide to form a precipitate of the corresponding metal or ammonium carbonate in which at least a portion of said high molecular weight coloured material is trapped, followed by separation of said precipitate.

By “reaction vehicle” herein is meant the diluent or solvent in which the chlorination reaction is performed. The term is meant to indicate that the vehicle may not fully dissolve all the components of the reaction and product mixture.

The chlorination reaction to produce the feed stream that is the starting point for the method of the present invention can be carried out by a number of methods, such as those disclosed in EP 0043649. Depending on the chlorination reagent employed, a number of types of reaction vehicles may be used, and any reaction vehicle can be used that is stable under the chlorination conditions and that dissolves the starting materials, reagents, and products at least to some extent, for example aromatic hydrocarbons such as xylene or toluene; chlorinated hydrocarbons such as trichloroethane; or tertiary amides such as dimethylformamide (DMF).

Preferably, the chlorination is carried out as described in EP 0409549. In that case, a tertiary amide is the reaction vehicle used in the chlorination reaction, and can be any tertiary amide that is stable under the chlorination conditions and that dissolves the starting materials, reagents, and products at least to some extent. The tertiary amide is typically dimethylformamide (DMF).

The removal of the reaction vehicle can be carried out by means known in the art, such as distillation, distillation under reduced pressure, steam distillation, steam stripping, or by use of an agitated thin film drier or spray drier. When the reaction vehicle is a tertiary amide, it is preferred that the removal of the reaction vehicle is carried out by steam stripping. Such steam stripping can be carried out as described in EP 0708110.

Furthermore, the isolation of the sucralose in the final step of the method of the present invention will also usually be carried out as described in EP 0708110.

The sucralose-6-acylate can be any acylate that serves to protect the 6-hydroxy group during the chlorination reaction. It is preferably an aliphatic or carbocyclic aromatic acylate, more preferably a benzoate or acetate, and most preferably an acetate.

For convenience, the characterising step in the above statement of invention will herein be referred to as the “precipitation step”. The metal or ammonium hydroxide used in said precipitation step can be any metal or ammonium hydroxide whose carbonate is at least partially insoluble under the conditions employed. It will be appreciated that the presence of a reaction vehicle such as a tertiary amide renders the metal carbonate less soluble than if the system were purely aqueous, and therefore a wider variety of metals can be employed than would otherwise be the case. However, the metal hydroxide is preferably an alkali or alkaline earth metal hydroxide, more preferably calcium hydroxide or sodium hydroxide. The precipitation step is preferably carried out at a pH of from 5 to 12 and a temperature of from 0 to 90° C., more preferably at a pH of from 6 to 10 and a temperature of from 25 to 80° C.

During the precipitation step, the hydroxide is added to the stream with addition of CO₂ gas. The CO₂ gas can be added simultaneously with the hydroxide, or the hydroxide can be added first, with subsequent addition of the CO₂ gas. This results in formation of a carbonate, which is insoluble or poorly soluble under the conditions, and therefore forms a precipitate. Colour removal is achieved by the entrapment of high molecular weight coloured material in this precipitate.

The addition of the hydroxide and the CO₂ gas preferably takes place over a period of from 15 to 120 minutes, more preferably over from 20 to 40 minutes.

The CO₂ gas need not be pure CO₂ gas. The CO₂ gas is preferably from 60 to 99% pure, more preferably from 80 to 95% pure, most preferably approximately 90% pure.

The amount of hydroxide added depends on the amount of high molecular weight coloured material present in the reaction stream, and can be determined by the person skilled in the art. Typically the hydroxide is added in an amount of 0.1 to 10% w/v of the reaction stream, more preferably 0.5 to 6% w/v of the reaction stream.

The amount of CO₂ gas added is preferably substantially stoichiometric relative to the hydroxide used, for conversion of the hydroxide into the corresponding carbonate.

The precipitate which thus forms is removed by any suitable technique, for example by a filtration technique, such as a rotary vacuum filtration apparatus, a pressure filter apparatus, or a gravity filter apparatus, or by a non-filtration technique, such as a centrifuge or a cyclone, or by decantation.

The deacylation is carried out by treatment with a base at a pH of from 8 to 14 and a temperature of from 0 to 60° C., preferably at a pH of from 10 to 12 and a temperature of from 0 to 40° C.

The base is preferably a metal or ammonium hydroxide, more preferably an alkali or alkaline earth metal hydroxide, more preferably calcium hydroxide or sodium hydroxide.

According to the present invention, there are two orders in which the steps can be carried out, namely deacylation, precipitation, steam stripping, or precipitation, stripping, deacylation. In contrast to the teaching of EP 0708110, in the present invention it is preferred to perform the deacylation first. Otherwise, partial deacylation can in any case take place during the precipitation step. Also, sucralose-6-acylates have a greater tendency to become included in the precipitate (and thus be lost) than sucralose itself, so the yield is reduced when the deacylation is performed last.

As stated above, according to EP 0708110, it is preferred not to perform the deacylation before the steam stripping because decomposition of the tertiary amide occurs under the deacylation conditions. Surprisingly, however, according to the present invention, it has been found that, provided that the reaction conditions are controlled carefully, deacylation can be achieved with minimal decomposition of tertiary amide. Therefore, according to the present invention, especially when deacylation is performed first, the deacylation is preferably performed under pH of from 10 to 13.5, more preferably from 10 to 12, and most preferably from 10.5 to 11.2, at a temperature of from 60 to 0° C., more preferably from 40 to 0° C., and most preferably from 35° C. to 25° C., the higher pH being used with the lower temperature and vice versa.

The deacylation reaction can be conveniently monitored by HPLC. For optimum yields, it is important to monitor the progress of the deacylation reaction, and neutralise the reaction mixture when the reaction is complete. The pH of the reaction mixture should be adjusted to from 6 to 8.5, preferably approximately 7.5. The reaction mixture can conveniently be neutralised using dilute hydrochloric acid, or using citric acid or acetic acid. Alternatively, and particularly conveniently when the deacylation is to be immediately followed by the precipitation step, the reaction mixture can be neutralised with gaseous carbon dioxide.

It will be appreciated that, in the present invention, the same metal hydroxide can in principle be used in the deacylation step and in the precipitation step, and that this leads to a simplicity of operation, especially when the precipitation step immediately follows the deacylation step. This can be a useful way of performing the present invention, particularly when calcium hydroxide or sodium hydroxide are used in both steps.

However, the present inventors have found that the pH in the deacylation step is easier to control when sodium hydroxide is employed than when calcium hydroxide is employed. Furthermore, the precipitation step is more effective when calcium hydroxide is employed than when sodium hydroxide is employed, and therefore it is most preferred to use sodium hydroxide in the deacylation step and calcium hydroxide in the precipitation step.

The invention will now be illustrated by means of the following examples, it being understood that these are intended to explain the invention, and in no way to limit its scope.

### EXAMPLES

### General

The feed stream came from the chlorination of sucrose-6-acetate with phosgene/dimethylformamide, after quenching with sodium hydroxide solution. Such a feed stream can be produced, for example, by the methods disclosed in EP 0 409 549. The typical composition of the feed stream from the chlorination reaction, which was used in these studies, was as follows:

| Description | % of total |
|---|---|
| Water | 49% |
| Dimethylformamide | 32% |
| Sodium chloride | 8% |
| Dimethylammonium hydrochloride | 4% |
| Sucralose-6-acetate | 3% |
| Sodium acetate | 1% |
| Others | 3% |

### Example 1

### Deacetylation with Sodium Hydroxide, Precipitation with Calcium Hydroxide/CO₂ pH Adjustment with Dilute Aqueous Hydrochloric Acid

### Example 1a

### High Lime

527 g of the above feed stream was continuously adjusted to pH 10.5 at 40° C. over a period of 4 hrs by dropwise addition of a total of 59 g of 27% NaOH solution. The progress of the deacetylation reaction was monitored using HPLC. When the deacetylation reaction was complete, the pH of the reaction mixture was lowered to pH 7 by adding 24.5 g of 20% HCl solution, over a period of 15 minutes.

523 g of the above deacetylated mixture was heated to 80° C. at pH 7. CO₂ gas was bubbled into this mixture over a period of 40 minutes. Simultaneously, an aqueous dispersion of freshly slaked CaO in water (16.7% dry solids) was added to the mixture, with the pH being controlled between 6 and 8. The total amount of slaked lime added was 70 g @ 16.7 wt %.

The final mixture had a muddy appearance due to the calcium carbonate co-precipitating with the coloured material.

This mixture was filtered to give a filter cake which was intensely coloured, and a filtrate that was less intensely coloured than the starting feed stream.

### Example 1b

### Low Lime

The procedure of Example 1a was repeated exactly, except using less slaked lime. In this case, 7 g @ 16.7 wt % of freshly slaked CaO in water was used. Again, calcium carbonate co-precipitated with the coloured material.

This mixture was filtered to give a filter cake which was intensely coloured, and a filtrate that was less intensely coloured than the starting feed stream.

Examples 1a and 1b show the range of proportions in which calcium hydroxide can be added in the precipitation step.

### Example 2

### Deacetylation with Sodium Hydroxide, Precipitation with Calcium Hydroxide/CO₂ pH Adjustment with Carbon Dioxide

### Example 2a

### High Lime

411.72 g of the feed stream as used in Example 1 was continuously adjusted to pH 10.5 @ 40° C. over a period of 4 hrs by drop wise addition of a total of 45.14 g of 27% NaOH solution.

When the deacetylation reaction was complete, as indicated by HPLC analysis, the reaction mixture was neutralised by bubbling CO₂ gas (100% pure) at a flow rate of 1-2 litres per minute until the pH reached between 7.2-7.5, which took about 30 minutes.

456.86 g of the above deacetylated mixture was heated to 80° C. at pH 7.2-7.5. CO₂ was bubbled into this mixture over a period of 40 minutes. Simultaneously, an aqueous dispersion of freshly slaked CaO in water (16.7% dry solids) was added to the mixture, with the pH being controlled between 6-8. The total amount of slaked lime added was 70 g@ 16.7%

As in Example 1, the final mixture had a muddy appearance due to the calcium carbonate co-precipitating with coloured material.

This mixture was filtered to give a filter cake which was intensely coloured, and a filtrate that was less intensely coloured than the starting feed stream.

### Example 2b

### Low Lime

The procedure of Example 2a was repeated exactly, except using less slaked lime. In this case, approx 7.0 g @ 16.7 wt. % was used.

This mixture was filtered to give a filter cake which was intensely coloured, and a filtrate that was less intensely coloured than the starting feed stream.

Examples 2a and 2b show the range of proportions in which calcium hydroxide can be added in the precipitation step.

### Example 3

### Use of Magnesium Hydroxide in Deacetylation and Precipitation

587 g of the feed stream as used in Example 1 was continuously adjusted to pH 10.5 @ 40° C. over a period of 30 minutes by dropwise addition of a total of 129.33 g of 23% Mg(OH)₂ solution. This raised the pH to 8.95. Then a total of 67.71 g of 23% sodium hydroxide solution was added for 2.5 hrs keeping the pH at 10.1 to 10.2. (Using magnesium hydroxide alone, the pH cannot easily be raised high enough to affect deacetylation).

When the deacetylation reaction was complete, as indicated by HPLC analysis, the reaction mixture was neutralised by bubbling CO₂ gas (100% pure) at a flow rate of 1-2 litres per minute over a period of 1.5 hrs. When the pH reached between 7.6 then enough CO₂ had been added.

Again, the final mixture had a muddy appearance typical of what was seen in Examples 1 and 2 due to co-precipitation of coloured material. This material was filtered to give 298.36 of cake and 446.97 g of filtrate.

### Example 4

### Use of Sodium Hydroxide in the Precipitation Step

470.1 g of a stock material consisting of the feed stream as used in Example 1 which had been deacetylated with NaOH and pH adjusted to 5.53 with dilute HCl was used as the feed. The pH was adjusted to pH 10.5 by adding 59.3 g of 10% NaOH solution at 40° C. CO₂ was bubbled into this mixture for 1 hr at 40° C. During this time the pH reduced to 7.4. The product was filtered through a filter cloth to give a cake of 3.94 g wet cake (1.67 g of dry cake) and a filtrate of 480.57 g.

### Example 5

### Use of Calcium Hydroxide for Both Deacetylation and Carbonatation

506.84 g of the feed stream as used in Example 1 was heated to 40° C. and agitated. 76.02 g of 23% freshly slaked lime was added. The pH was 10.2. After 2.5 hrs, HPLC analysis showed that the deacetylation was complete. CO₂ was then bubbled into the reaction mixture to reduce the pH to 5.8-6.

Then, 4.3 g of 22% freshly slaked lime solution was added, then CO₂ was bubbled in over a period of 30 minutes to form a mixture with a muddy appearance. This mixture was then filtered to recover a coloured cake (20 g wet) and a filtrate of 534 g.

### Example 6

### Large Scale

350 gallons of the feed stream as used in Example 1 was continuously adjusted to pH 11.1 at a temperature of 91.5° F. over a period of 8 hrs by addition of a total of 70 gallons of 12% NaOH solution.

The progress of the deacetylation reaction was monitored using HPLC. When the deacetylation reaction was complete, the reaction mixture was then neutralised by adding 24.5 gallons of 20% HCl solution over a period of 15 minutes.

444.5 gallons of the above deacetylated mixture was heated to 45° C. at pH 7.5, and CO₂ gas was bubbled into this mixture over a period of 90 minutes. Simultaneously, an aqueous dispersion of freshly prepared hydrated lime, Ca(OH)₂, in water (16. % dry solids) was added to the mixture, with the pH being controlled between 6 and 8. The total amount of slaked lime added was 41 gallons @ 16 wt %. [This is 50 lb of hydrated lime mixed with 31.5 gallons of water]. The final mixture had a muddy appearance due to the calcium carbonate co-precipitating with coloured material.

This mixture was filtered using a Putsch press to give a filter cake which was intensely coloured, and a filtrate that was less intensely coloured than the starting feed stream.

### Example 7

### Deacetylation/Carbonatation

i) Deacetylation

A jacketed reaction tank was charged, using manual valves, from a feed tank with a pre-determined volume of the feed stream described above. The reaction tank recirculation pump was started. After 5 minutes, a sample of the starting material was taken. The reactor was manually controlled at 33° C. by using low pressure steam or cooling water on the jackets. Caustic was dosed into the reactor using a diaphragm pump to control the pH to 11.1±0.1. A sample was taken of the reaction mixture hourly. The reaction conditions were maintained for 8 hours and then a separate diaphragm pump was used to neutralise the mixture using HCl.

ii) Carbonatation

The deacetylated reaction product was heated to 55° C. by manually adjusting the low pressure steam inlet valve to the jacket. The CO₂ inlet valve was opened and a flow rate of 2 lbs/h measured on a flowmeter was sent to the sparger. The pH was monitored and after 5 minutes of CO₂ flow (reaction mixture pH 7.5-8.0) the 16% lime solution dosing started. The pH was balanced in the reactor so that it was between 8.0 and 8.5. The CO₂ flow was increased to >9 lbs/h so that the dosing could be completed in a reasonable amount of time. When all the lime had been dosed, the CO₂ was reduced back to 2 lbs/h and allowed to run for 20 minutes or until the pH was at 8.0.

iii) Filtration

With the reaction tank recirculation pump in continuous operation, a peristaltic pump was used to pump the carbonatated product from the reaction tank to the pilot Putsch filter press. The filtration was run until 30 psi pressure was reached, the pressure limit of this particular set-up. At this pressure, the chambers were approximately 80% full with cake which is standard for full scale operations to enable efficient squeezing of the cake. The cake was squeezed first at 29 psi until no more permeate flowed. This was then increased to 73 psi until no more permeate flowed. Water was then introduced into the press and 40 litres (4× cake mass) was allowed to pass through the cake. The washed cake was then pressed a third time at 100 psi before a one minute, 50 scfm N₂ blow down dried the cake.

iv) Stripping a condenser was connected to a stripping column. To start up the column, a cooling water valve was opened to the condenser. Then a steam control valve was opened to the column and controlled on a flow indicator to 30% (nominal units). This equated to approximately 40 lbs/hr although this was subject to variation due to pressure fluctuations in the 50 psi header. When the column had warmed up, the feed was pumped in at approximately 100 ml/min using a peristaltic pump. The top product valve was manually controlled to keep a small level in the condenser and hence regulate the temperature inside the column. The bottom product valve was also regulated to maintain a level and prevent downward steam flow. The column was operated for runs of one hour to eight hours and the resulting fouling and stripping performance evaluated against the other feeds.

Results

Colour Removal

Analyses of absorbance on the original feed, deacetylated samples and carbonatated samples shows the carbonatation process removing on average 40% of the colour present in the feed. Data is shown in the following Table:

TABLE 2

| | Integrated Abs Area | | | Carb. % |
|---|---|---|---|---|
| Batch | Original feed | Deacetylated | Carbonatated | removal on Deac. |
| 1 | 2660 | 1990.5 | 1050.5 | 47% |
| 2 | 2175 | 1972.2 | 1418.5 | 28% |
| 3 | 2952.2 | 2454.1 | 1251.8 | 49% |
| 4 | 3109.2 | 2533.6 | 1370.1 | 46% |
| 5 | 2554 | 2311.1 | 1116.9 | 52% |
| B20 | 3262 | 2241.4 | 1533.9 | 32% |
| B21 | 4269.4 | 3357.1 | 2204.6 | 34% |

Filtration

Filtration of the carbonatated product was performed on a pilot Putsch unit. The filtered solids from each carbonatation batch were washed in situ on the Putsch press with town water to minimise DMF and sucralose losses. Sucralose losses on the filter cake were calculated as <0.03%. The DMF losses were <0.02%.

Stripping

On each batch, deacetylated and carbonatated material was fed to the pilot stripper. The fouling characteristics of each material was observed by the naked eye. In all cases the carbonatated and filtered product produced less fouling than its corresponding deacetylated feed. This in turn was superior to the original feed. The observations were based on quantity and type of fouling.

1. A method for the production of sucralose from a feed stream resulting from the chlorination of a sucrose-6-acylate in a reaction vehicle, said feed stream comprising a sucralose-6-acylate, the reaction vehicle, and by-products including high molecular weight coloured material, said method comprising:
deacylation of the sucralose-6-acylate by treatment with a base to afford sucralose, and, before or after said deacylation,
removal of the reaction vehicle and
isolation of the sucralose characterised in that
immediately before the removal of the reaction vehicle, the reaction stream is subjected to a precipitation step comprising treatment with a metal or ammonium hydroxide and carbon dioxide to form a precipitate of the corresponding metal or ammonium carbonate in which at least a portion of said high molecular weight coloured material is trapped, followed by separation of said precipitate.

2. A method according to claim 1, wherein the removal of the reaction vehicle is performed after the deacylation, so that the steps are carried out in the order: deacylation, precipitation, removal of reaction vehicle.

3. A method according to claim 1, wherein the removal of the reaction vehicle is performed before the deacylation, so that the steps are carried out in the order: precipitation, removal of reaction vehicle, deacylation.

4. A method according to claim 1, wherein the removal of the reaction vehicle is performed by steam stripping, or by use of an agitated thin film drier or spray drier.

5. A method according to claim 1, wherein the sucralose-6-acylate is sucralose-6-benzoate or sucralose-6-acetate.

6. A method according to claim 5, wherein the sucralose-6-acylate is sucralose-6-acetate.

7. A method according to claim 1, wherein the reaction vehicle is a tertiary amide.

8. A method according to claim 7, wherein the tertiary amide is dimethyl formamide (DMF).

9. A method according to claim 1, wherein said metal or ammonium hydroxide is an alkali or alkaline earth metal hydroxide.

10. A method according to claim 9, wherein said metal or ammonium hydroxide is an alkaline earth metal hydroxide.

11. A method according to claim 10, wherein said alkaline earth metal hydroxide is calcium hydroxide.

12. A method according to claim 9, wherein said metal or ammonium hydroxide is an alkali metal hydroxide.

13. A method according to claim 12, wherein said alkali metal hydroxide is sodium hydroxide.

14. A method according to claim 1, wherein said metal or ammonium hydroxide is ammonium hydroxide.

15. A method according to claim 1, wherein the base used in the deacylation is a metal or ammonium hydroxide.

16. A method according to claim 15, wherein said base is an alkali or alkaline earth metal hydroxide.

17. A method according to claim 16, wherein said base is an alkali metal hydroxide.

18. A method according to claim 17, wherein said base is sodium hydroxide.

19. A method according to claim 16, wherein said base is an alkaline earth metal hydroxide.

20. A method according to claim 19, wherein said base is calcium hydroxide.

21. A method according to claim 2, wherein said metal or ammonium hydroxide and said base are both the same compound.

22. A method according to claim 21, wherein said compound is an alkali or alkaline earth metal hydroxide.

23. A method according to claim 22, wherein said compound is sodium hydroxide or calcium hydroxide.

24. A method according to claim 1, wherein said deacylation is carried out at a pH of from 8 to 14 and a temperature of from 0 to 60° C.

25. A method according to claim 24, wherein said deacylation is carried out at a pH of from 10 to 12 and a temperature of from 0 to 40° C.

26. A method according to claim 1, wherein said precipitation step is carried out at a pH of from 5 to 12 and a temperature of from 0 to 90° C.

27. A method according to claim 26, wherein said precipitation step is carried out at a pH of from 6 to 10 and a temperature of from 25 to 80° C.

28. A method according to claim 1, wherein the separation of the precipitate is performed by filtration.

29. A method according to claim 28, wherein the filtration is performed using a rotary vacuum filtration apparatus, a pressure filter apparatus, or a gravity filter apparatus.

30. A method according to claim 1, wherein the separation of the precipitate is performed by a non-filtration technique.

31. A method according to claim 30, wherein the separation of the precipitate is performed using a centrifuge, a cyclone, or by decantation.

32. A method according to claim 2, wherein the pH of the reaction stream is reduced after the deacetylation and before the precipitation step by the addition of acid.

33. A method according to claim 32, wherein said acid is dilute aqueous hydrochloric acid, acetic acid, citric acid, or carbon dioxide.
